(19) United States
(12) Patent Application Publication
(10) Pub. No.: US 2017/0101460 A1
(43) Pub. Date: Apr. 13, 2017

(54) TRANSGENIC ANIMALS CAPABLE OF PRODUCING HUMANIZED IGE AT MUCH HIGHER LEVELS THAN MOUSE IGE

(71) Applicant: ALLERMABS CO. LTD., Taipei City (TW)

(72) Inventors: Alfur Fu-Hsin Hung, Taichung City (TW); Donic Chien-Sheng Lu, Kaohsiung City (TW); Tse-Wen Chang, Taipei (TW)

(73) Assignee: ALLERMABS CO. LTD., Taipei City (TW)

(21) Appl. No.: 15/110,555

(22) PCT Filed: Jan. 12, 2015
(86) PCT No.: PCT/CN2015/070540

§ 371 Date: Jul. 8, 2016

Related U.S. Application Data
(60) Provisional application No. 61/925,836, filed on Jan. 10, 2014.

(51) Int. Cl. C07K 16/00; C12N 5/16; C12N 5/26; A01K 67/027

(52) U.S. Cl. CPC C07K 16/00; A01K 67/0278; C12N 5/163; C12N 5/166; C12N 2510/02; A01K 2207/15; A01K 2217/052; A01K 2217/072; A01K 2217/15; A01K 2227/105; A01K 2267/01; C07K 2317/24

(57) ABSTRACT

The transgenic non-human animals are constructed, in whose genome the coding sequences of one of the animal's endogenous immunoglobulin Cγ constant regions are replaced by human immunoglobulin Cε constant region coding sequences. The transgenic animal is mouse, in whose genome the Cγ1 constant regions are replaced by the human immunoglobulin Cε constant regions and the Cκ constant region is replaced by the human immunoglobulin Cκ constant region. The transgenic mouse yields humanized IgE-secreting B cells and antigen-specific humanized IgE after immunization. The transgenic animals are employed to prepare serum containing humanized IgE, antiserum containing antigen-specific humanized IgE, and monoclonal antigen-specific humanized IgE antibodies by hybridoma and other technologies.

### BACKGROUND AND RATIONALE

IgE plays a central role in mediating type I hypersensitivity reactions that are responsible for causing allergic diseases, including allergic asthma, allergic rhinitis, atopic dermatitis, and others. Allergic reactions result from the immune response to harmless environmental substances, such as dust mites, tree and grass pollens, certain foods, insect stings, and others. In sensitized individuals, the immune system produces IgE specific to the antigens the persons are sensitized to. In an allergic reaction, the antigen inhaled, ingested, or taken in through the skin by a sensitized person binds to IgE on the surface of basophils and mast cells, thus causing the cross-linking of the IgE and the aggregation of the underlying receptor of IgE.Fc (the type I IgE.Fc receptor, or FcεRI), leading to the release of pharmacologic mediators, such as histamine, leukotrienes, tryptase, cytokines and chemokines from those inflammatory cells. The release of those mediators from mast cells and basophils causes the various pathological manifestations of allergy.

The genes encoding the classes and subclasses of immunoglobulins, including the constant regions of μ, δ, γ, α, and ε chains, are clustered in a stretch of coding regions and introns in one chromosome in the respective genome of human, mouse, or other mammals. In both humans and mice, there are several γ subclasses and one functional c subclass. The expression and stability of Ig classes and subclasses are regulated by a host of regulatory factors and receptors expressed by B and T lymphocytes and other cell types and by a large array of segments/elements of DNA in the genes of the immunoglobulins.

Among the five Ig classes, IgE is generally present in minute concentrations in serum in non-atopic persons, generally ranging from 10 to 400 ng/ml (Hellman 2007). The concentrations of IgE in mice, rats, rabbits, and other mammals are also very low compared to IgG, IgM, and IgA. In the preparation of mouse or rat hybridomas, which secrete monoclonal antibodies specific for the antigens used in immunizing the animal hosts, hybridomas secreting IgE are extremely rare and very difficult to obtain. In contrast, IgG is the dominant plasma Ig class with serum concentrations normally in the range of 8˜16 mg/ml (Hellman 2007). In preparing mouse or rat hybridomas, IgG is the dominant class of antibodies the hybridomas secrete.

Hybridomas secreting hapten-, ovalbumin-, or allergen component-specific mouse IgE can be prepared by fusing splenocytes from antigen-immunized mice or rats with a mouse myeloma cell line by a conventional cell fusion technique (Bottcher 1980, Bohn 1982, Akihiro 1996, Hanashiro 1996, Susanne 2003). Typically not a single antigen-specific IgE hybridoma can be identified even from several hundreds of hybridoma clones, most of which secret IgG isotypes. The Yu's group constructed an IgE knock-in mouse line in which the DNA sequence encoding mouse Ig γ1 constant region was replaced by the sequence encoding mouse Ig c constant region (Yu 2013). Total serum IgE levels in those mice increased about ten folds as compared to those in the wild type mice. The number of IgE-expressing lymphocytes isolated from the spleen of a knock-in mouse also significantly increased under the stimulation with lipopolysaccharide (LPS) and Interleukin-4 (IL-4) in vitro. The Zarrin's group constructed an SiKI mouse line in which the switch region of Ig ε heavy chain gene was substituted by the switch region of mouse Ig μ heavy chain gene (Zarrin, 2013). A switch region is a conserved DNA sequence upstream of Ig heavy chain gene and plays a role in Ig isotype switching. In using the SμKI mice to prepare hybridomas, the percentage of IgE-secreting hybridomas and the ratio of IgE to IgG hybridoma numbers increased when compared to results using the wild type mice.

Prior to our invention, there has not been a scientific paper or patent disclosure that describes the preparation of hybridomas by the conventional procedure of fusing mouse spleen cells with mouse myeloma cells and such hybridomas secrete human or “humanized” IgE that is specific to a defined protein component. Rare IgE-expressing B lymphocytes in human peripheral blood mononuclear cells and the low cell fusion efficiency of human B lymphocytes with human myelomas or lymphoma cell lines have hindered the preparation of hybridomas secreting human IgE. The Hakamata's group prepared a mite extract-specific human IgE hybridoma by using in vitro cytokine-activated and mite-extract-treated lymphocytes isolated from healthy donors (Hakamata 2000). The produced IgE mAb reacts with the mite extract rather than with a defined protein component (Hakamata 2000). In addition, a hybridoma secreting Der p 2-specific chimeric or “humanized” IgE was prepared by a gene transfection procedure (Aalberse 1996). In this study, a recombinant gene containing DNA segments encoding mouse heavy chain variable region specific for Der p 2 joined with human E constant region and a geneticin-resistant protein was transfected into a mouse Der p 2-specific hybridoma variant, which had already lost its γ2b heavy chain gene. After drug selection of transfected cells and reactivity tests for survival clones, the humanized IgE hybridoma specific to Der p 2 was prepared (Aalberse 1996).

### SUMMARY OF THE INVENTION

Transgenic non-human animals are disclosed which are capable of producing abundant polyclonal “humanized” IgE. In this invention disclosure, “humanized” IgE represents that the constant region of the immunoglobulin c of the IgE, encompassing CH1, CH2, CH3, CH4, M1, and M2, is human and variable region is the animal's own. M1 and M2, which are respectively encoded by two “membrane exons” in the ε gene, represent two contiguous peptide segments that form the membrane-anchor peptide of 69 amino acid residues extending from the C-terminal of membrane-bound ε heavy chain (mε). In some embodiments, the humanized IgE also include a form of IgE, in which the constant regions of both ε heavy chain and κ light chain are human and the variable regions of the heavy and light chains are the animal's own. The transgenic animals are mouse, rat, and rabbit, for which methods for genetic manipulation and alteration are established. Thus, for these transgenic animals, the coding sequences of CH1, CH2, CH3, M1, and M2 for one of the Cγ immunoglobulin gene are replaced by the corresponding coding sequences of human Cε immunoglobulin gene. It is noted that a γ chain has only 3 CH domains and also has a C-terminal membrane anchor peptide that is encoded by two membrane exons.

A preferred embodiment of this invention is mouse and the Cγ gene chosen is Cγ1. For further enhancing the “humanness” antigenic property of the humanized IgE, the transgenic mouse strain is crossed with a transgenic mouse strain, in whose genome the coding region of the constant region of the mouse κ chain is replaced by the corresponding coding segment of human κ chain, to obtain the homozygous transgenic mouse strain that harbor human Cε and Cκ constant region genes.

The invention also pertains to the applications of the transgenic animals constructed as described above in producing serum containing humanized IgE, antigen-specific humanized IgE, and hybridomas producing antigen-specific humanized IgE. For preparing antiserum containing antigen-specific IgE and for preparing hybridomas secreting antigen-specific humanized IgE in transgenic mice or rats, the animals are immunized with the specified antigens, such as dust mites of particular strain or region, pollens of a particular tree or grass, shed dander of cats, or isolated antigens of certain foods, to boost the proportion of antigen-specific humanized IgE in total IgE. The serum containing polyclonal humanized IgE, antisera containing antigen-specific humanized IgE, or the antigen-specific humanized monoclonal IgE can be applied for various immunoassays for measuring IgE or antigen-specific IgE in the sera of patients with IgE-mediated allergy.

### DETAILED DESCRIPTION OF THE INVENTION

### 1. Altering the Relative Abundance of Immunoglobulin Isotypes

The immunoglobulin heavy chain gene locus (IGHC) contains in one cluster of the genes encoding the constant regions of all of the classes and subclasses of heavy chains, including μ chain of IgM, β chain of IgD, and γ chain of IgG, and a chain of IgA, and ε chain of IgE. In both human and mouse, the γ class has four subclasses and the α class has two subclasses. In human genome, the IGHC is arranged in the order of μ-β-γ3-γ1-α1-γ2-γ4-ε-α2, and in the mouse genome, IGHC is arranged in the order μ-δ-γ3-γ1-γ2b-γ2a (or γ2c)-ε-α. The gene elements encoding each of the subclasses is separated from the neighboring subclass by the switch (S) regions involved in class switch recombination (CSR).

The immune-competent resting B lymphocytes bear surface membrane-bound IgM and IgD (mIgM and mIgD). Upon initial antigen stimulation, the first antibodies produced by the lymphocytes are of the IgM class. With continual or repeated antigen stimulation, the activated B lymphocytes expand, differentiate, and secrete antibodies toward the antigens. One important aspect of this antibody response is that the B cells undergo isotype-switching from originally IgM production to the production of another isotype. The regulation and the determination of isotypes are mediated by a network of cytokines, chemokines, transcription activators, and negative regulators. Following antigen stimulation, signaling pathways recruit those factors which regulate the expression of germ line transcripts and the switch regions of the individual genes (Chaudhuri and Alt 2004; Stavnezer and Amemiya 2004; Pan-Hammarstroem et al. 2007). CSR that effectuates the change in antibody class is a deletional recombination where the constant region gene of the heavy chain Cμ is replaced by a downstream C_{H} gene and the intervening sequences are excised as circular DNA. CSR is initiated by activation-induced deaminase acting within the S region, which is followed with double strand breaks, DNA damage response/repair pathways and nonhomologous end joining (Chaudhuri and Alt 2004). The Ig of different class and subclass is expressed at different levels. In general, IgG, IgA, and IgM are expressed at much higher levels than IgD and IgE. And between IgD and IgE, the latter is still much lower. In addition to the different levels of production among the different classes, the turnover rate of free Ig and the stabilization of each Ig class by its receptor contribute to the overall turnover kinetics, the abundance, and half-life of the Ig class.

The present invention pertains to genetically altering an animal, so that the IgE in the altered animal becomes humanized IgE and its production is much higher than the IgE in an unaltered animal host. For achieving this, a mouse, rat, or rabbit is used, because genetic alteration of the antibody genes in these animals can be achieved with existing tools of molecular biology and embryonic stem cell manipulation, and the information concerning the immunoglobulin gene complexes in these animals. Furthermore, among these animals, mouse is a good choice because the time for reproduction is short and the tools for preparing transgenic strains are well established.

To increase the overall IgE levels, the coding sequences for the constant region of one of Cγ immunoglobulin, such as Cγ1, which is expressed at high levels, is replaced by the coding sequence for the constant region of human Cε. In doing so, the regulatory sequences in the promoter and the S regions of the mouse own Cγ gene are kept, so that the control of expression of the knock-in human Cε may also achieve high expression. It is noted that since human IgE is not recognized by mouse FcεRI, the transgenic mice should not have adverse conditions even they produce large quantities of humanized IgE.

### 2. Construction of a Chimeric Transgene Comprising Human Cε Coding Sequences Replacing the Mouse Cγ1 Coding Sequences in Mouse Immunoglobulin Heavy Chain γ Gene Locus (mIGHG)

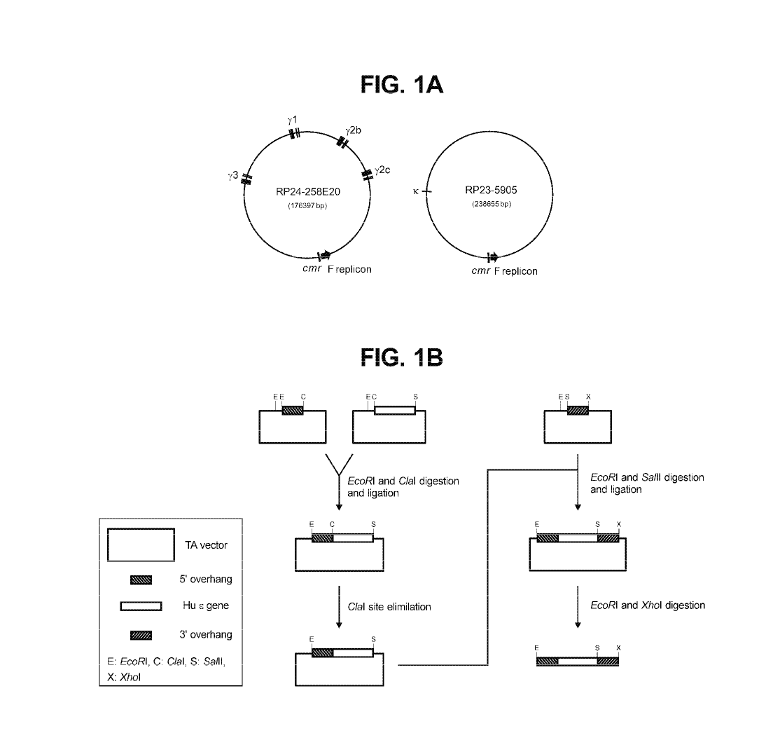

The replacement is achieved via homologous recombination between a designed construct and a mouse BAC clone containing the mouse IGHG locus (Clone ID RP24-258E20, FIG. 1A). The construct can be generated by PCR amplification incorporating the coding regions of human Cε CH1-CH2-CH3-CH4-M1-M2, flanked at either end with 2,000 bp each of the mouse sequences upstream and downstream, respectively, of the mouse Cγ1 gene at the recombination sites. The homologous recombination can be performed in E. coli using the Red®/ET® Recombination methodology (Gene Bridges GmbH, Dresden, Germany). Specifically, the homologous recombination occurs in two steps. First, a counter selection marker rpsL-neo replaces the mouse Cγ1 coding region for CH1-H-CH2-CH3-M1-M2 and is incorporated between the mouse homologous arms (the 2,000 bp sequences described above). “H” represents the hinge region. Then, the counter selection marker is replaced with the human Cε region encoding CH1-CH2-CH3-CH4-M1-M2.

### 3. Construction of a Chimeric Transgene Comprising Human Cκ Coding Sequences Replacing the Mouse Cκ Coding Sequences in Mouse Immunoglobulin Light Chain κ Locus (IGKC)

A construct is designed with PCR amplification incorporating human Cκ coding sequences flanked at either end with 50 bp each of the mouse sequences in the noncoding region upstream and downstream, respectively, of the mouse Cκ gene at the recombination sites. The construct is then integrated into a mouse BAC clone containing the IGKC locus (Clone ID RPCI23-59O5, FIG. 1A) via Red®/ET® Recombination methodology in E. coli (Gene Bridges GmbH, Dresden, Germany). Again, the homologous recombination occurs in two steps. First, a counter selection marker rpsL-neo replaces the mouse Cκ coding region and is incorporated between the mouse homologous arms (the 50 bp sequences described above). Then, the counter selection marker is replaced with the human Cκ coding sequences.

### 4. Generation of Transgenic Mice Harboring the Chimeric Transgenes

The method for transgene transfer employs the embryonic stem cell (ES). ES cells are obtained from pre-implantation embryos cultured in vitro and fused with embryos. Transgenes can be efficiently introduced into the ES cells by electroporation, retrovirus-mediated transduction or other methods. The preferred method is electroporation. Such transformed ES cells can thereafter be combined with blastocysts from a nonhuman animal. The ES cells thereafter colonize the embryo and contribute to the germ line of the resulting chimeric animal.

Homologous recombination can also be used to introduce transgenes. Homologous recombination can be mediated by either RecE/RecT (RecE/T) or Red α/β. In E. coli, any intact, independently replicating, circular DNA molecule can be altered by RecE/T or Red α/β mediated homologous recombination with a linear DNA fragment flanked by short regions of DNA sequence identical to regions present in the circular molecule. Integration of the linear DNA fragment into the circular molecule by homologous recombination replaces sequences between its flanking sequences and the corresponding sequences in the circular DNA molecule.

The homologous recombination described in sections 3 and 4 above yield transgenes comprising modified mouse BAC clones harboring the human Cε coding sequences and Cκ coding sequences, respectively. Each transgene is then introduced via electroporation into embryonic stem cells of mouse strain C57BL/6 where homologous recombination of the transgene and the corresponding endogenous gene locus takes place. The colonies verified to contain successfully recombined transgenes are then injected into blastocysts of C57BL/6, which are subsequently transferred into the uterus of pseudopregnant mice of the C57BL/6J-c2J strain. The embryos are allowed to develop into chimeric mice, which are then monitored to produce transgenic mice as in the standard procedures listed above.

The transgenic mice harboring the human Cε coding region substituting mouse Cγ1 coding region and those harboring the human Cκ coding region substituting mouse Cκ coding region are then crossed to produce mice harboring both transgenes in place of the respective endogenous coding sequences. The resulted mouse strain that harbors both transgenes is used for the production of antigen-specific humaninzed IgE and hybridomas secreting antigen-specific humanized IgE.

### 5. Production of Antiserum Containing Antigen-Specific Humanized IgE and Hybridomas Secreting Antigen-Specific Humanized IgE

The transgenic mice resulted from the crosses as described in section 4 are used to generate antigen-specific humanized IgE and hybridomas secreting antigen-specific humanized IgE. Two examples of specific IgE production are: (i) antigens, such as dust mites, and weed, grass or tree pollens, and (ii) Geohelminth parasites, such as Necator americanus (human hookworm) and Trichurls suis (pig whipworm).

### Examples

### 1. Preparation of Recombination-Potent Bacterial Artificial Chromosome (BAC)-Bearing Bacteria and Replacing Mouse Cγ1-Encoding Gene with a Prokaryotic Selection DNA Cassette

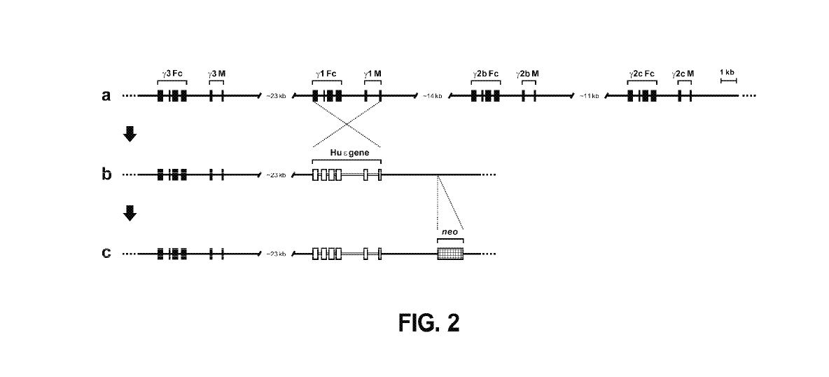

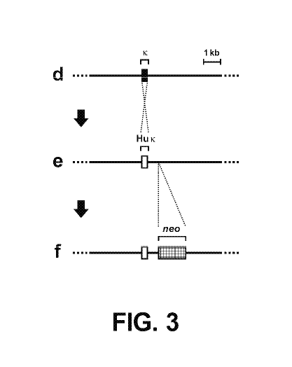

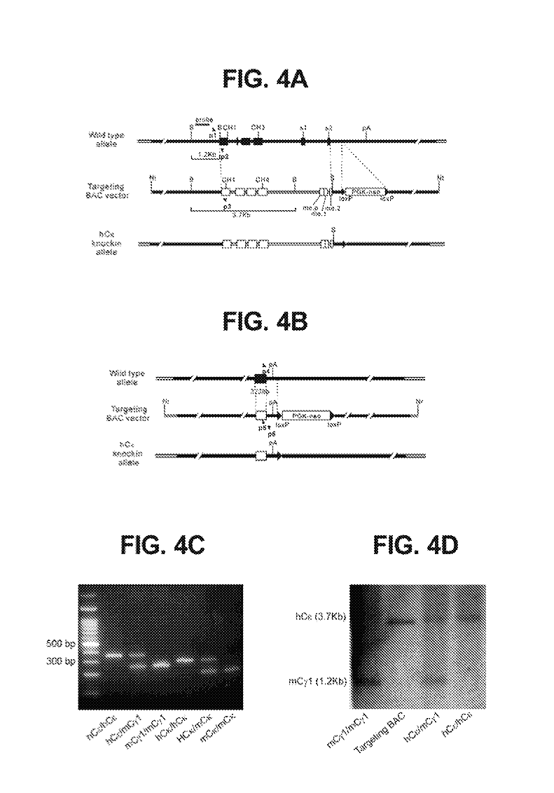

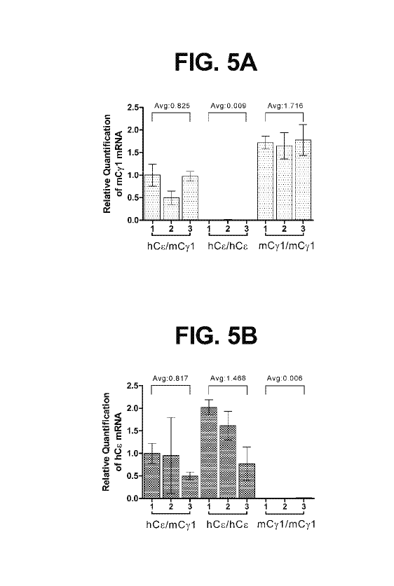

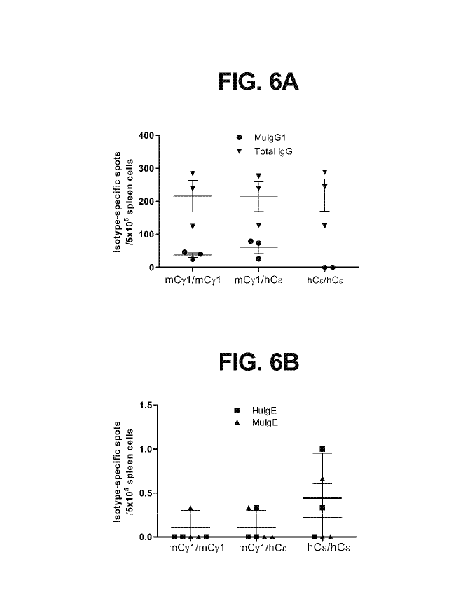

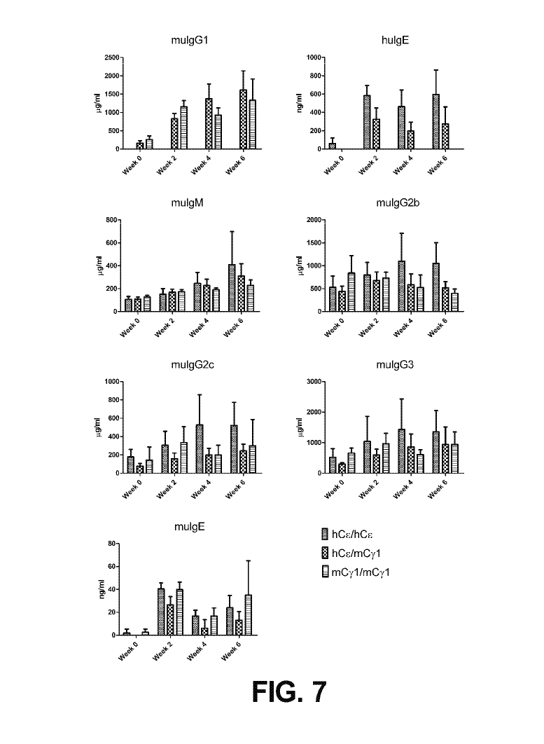

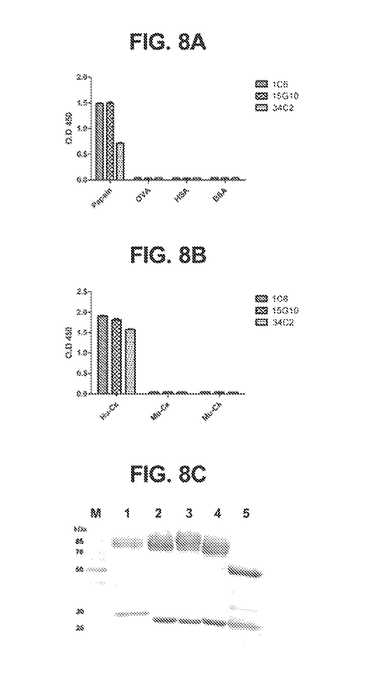

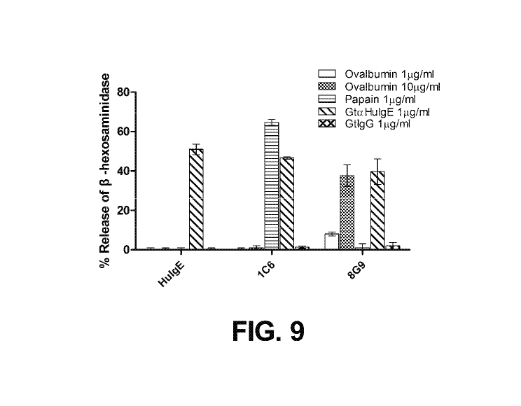

The bacterial clone carrying BAC RP24-258E20, which contains gene exons encoding mouse four Cγ heavy chains (FIG. 1A and FIG. 2, sequence a), was purchased from BACPAC Resources Center. The gene replacement was accomplished by using the Red/ET-based recombination system.

To prepare recombination-potent BAC-bearing bacteria, the pRed/ET plasmid DNA which encodes enzymatic proteins essential for mediating homologous recombination was delivered into the BAC-bearing bacteria. A single colony of BAC-bearing bacteria grown on LB agar with chloramphenicol and streptomycin was inoculated in 1 ml LB medium with antibiotics. After culturing at 37° C. overnight, the bacteria (30 μl) were added into 1.4 ml of LB medium with antibiotics and cultured at 37° C. for 2 hours. The bacteria were placed on ice followed by centrifugation at 11,000 rpm for 30 s and the supernatant was removed. The pellet was washed with 1 ml of chilled 10% glycerol and centrifuged to remove the supernatant. The pellet was resuspended in 20-30 μl of chilled 10% glycerol and placed on ice. The pRed/ET plasmid DNA (20 ng) was added into the bacteria and mixed briefly. The mixture was transferred into a chilled 1-mm electroporation cuvette and shocked at 1.8 kV, 200 ohms, and 25 μF for 4.5˜5.0 ms. The electroporation condition was used in the following examples. LB medium (1 ml) was added to resuspend the bacteria and then transferred into a culture vessel. The bacteria were cultured at 30° C. for 70 mins and 100 μl of cultured bacteria was spread onto an LB agar plate with chloramphenicol and tetracycline. The plate was incubated at 30° C. overnight for growth of pRed/ET plasmid DNA-carrying bacteria which were recombination-potent.

The mouse Cγ1-encoding gene in the recombination-potent BAC-bearing bacteria was replaced by a prokaryotic selection DNA cassette which contains a hybrid rpsL-neo gene that confers streptomycin-sensitive and kanamycin-resistant selection for transfected bacteria. A single colony of the recombination-potent BAC-bearing bacteria was inoculated in 1 ml of LB with chloramphenicol and tetracycline. After culturing at 30° C. overnight, 30 μl of cultured bacteria were added into 1.4 ml of LB medium with antibiotics followed by culturing at 30° C. for 2 hours. L-arabinose at final 10% was added into the culture bacteria with culturing at 37° C. for another 1 hour. The bacteria were placed on ice and then centrifuged at 11,000 rpm for 30 s to remove the supernatant. The pellet was then washed with 1 ml of chilled 10% glycerol and centrifuged to remove the supernatant. The pellet was then resuspended in 20-30 μl of chilled 10% glycerol and placed on ice. The DNA stretch containing the hybrid rpsL-neo gene flanked with two 50-bp DNA sequences corresponding to intronic sequences of the overhangs of mouse Cγ1-encoding gene (SEQ ID NO:1) was prepared by polymerase chain reaction (PCR) with specific primers (TABLE 1, primers G1_CH1-rpsL-neo+ and G1_M2-rpsL-neo-). The purified DNA product (100-200 ng) was added into the resuspended bacteria with brief mix. The mixture was transferred into a chilled 1 mm cuvette for electroporation. LB medium (1 ml) without antibiotics was added to resuspend the shocked bacteria and transferred into a culture vessel. The bacteria were cultured at 37° C. for 70 mins and 100 μl of the cultured medium was spread onto an LB agar plate containing chloramphenicol, kanamycin, and tetracycline. The plate was incubated at 30° C. overnight and the grown colonies were screened for identifying bacteria carrying rpsL-neo knock-in BAC by colony PCR with specific primers (TABLE 2, primers G1_CH1-up-sc+ and rpsL_sc−). Identified clones were grown onto an LB agar plate with antibiotics at 30° C. overnight.

1. A transgenic animal, in whose genome the gene segment encoding CH1-CH2-CH3-M1-M2 of one of the animal's endogenous immunoglobulins of Cγ is replaced by the gene segment encoding CH1-CH2-CH3-CH4-M1-M2 of human immunoglobulin Cε.

2. A transgenic animal of claim 1, in which the animal is a mouse, rat, or rabbit.

3. A transgenic animal of claim 1, in which the animal is a mouse and the Cγ is Cγ1.

4. A transgenic mouse of claim 3, in which the mouse is further crossed with a transgenic mouse, in whose genome the mouse's endogenous Cκ constant region coding sequence is replaced by the human immunoglobulin Cκ constant region coding sequences.

5. A method for producing serum or antigen-specific antiserum containing humanized IgE by using a transgenic animal, in whose genome the gene segment encoding CH1-CH2-CH3-M1-M2 of one of the animal's endogenous immunoglobulins of Cγ is replaced by the gene segment encoding CH1-CH2-CH3-CH4-M1-M2 of human immunoglobulin Cε; for the method of producing antigen-specific antiserum, the animal is immunized with the specific antigen.

6. A method for producing serum or antigen-specific antiserum containing humanized IgE of claim 5, wherein the transgenic animal is a mouse, rat, or rabbit.

7. A method for producing serum or antigen-specific antiserum containing humanized IgE of claim 5, wherein the animal is a mouse and the Cγ is Cγ1.

8. A method for producing serum or antigen-specific antiserum containing humanized IgE of claim 7, wherein the mouse strain is further crossed with a transgenic mouse strain, in whose genome the mouse's endogenous Cκ constant region sequence is replaced by the human immunoglobulin Cκ constant region sequence; the homozygous mouse strain with both transgenic human Cε and Cκ is used as the host for the production of serum or antigen-specific antiserum.

9. A method of preparing antigen-specific humanized IgE-secreting hybridomas by using the lymphocytes of a transgenic animal, in whose genome the gene segment encoding CH1-CH2-CH3-M1-M2 of one of the animal's endogenous immunoglobulins of Cγ is replaced by the gene segment encoding CH1-CH2-CH3-CH4-M1-M2 of human immunoglobulin Cε; the animal is immunized with the specific antigen.

10. A method of preparing antigen-specific humanized IgE-secreting hybridomas of claim 9, wherein the transgenic animal is a mouse, rat, or rabbit

11. A method of preparing antigen-specific humanized IgE-secreting hybridomas of claim 9, wherein the animal is a mouse and the Cγ is Cγ1.

12. A method of preparing antigen-specific humanized IgE-secreting hybridomas of claim 11, wherein the mouse strain is further crossed with a transgenic mouse strain, in whose genome the mouse's endogenous


SEQUENCE LISTING (49 sequences)

SEQ ID NO: 1 - DNA, 1419 bp, Artificial Sequence
  Heavy chain counter selection marker
tggagcccttccttgttacttcataccatcctctgtgcttccttcctcagggcctggtgatgatggcgggatcgttgtatatttcttgacaccttttcggcatcgccctaaaattcggcgtcctcatattgtgtgaggacgttttattacgtgtttacgaagcaaaagctaaaaccaggagctatttaatggcaacagttaaccagctggtacgcaaaccacgtgctcgcaaagttgcgaaaagcaacgtgcctgcgctggaagcatgcccgcaaaaacgtggcgtatgtactcgtgtatatactaccactcctaaaaaaccgaactccgcgctgcgtaaagtatgccgtgttcgtctgactaacggtttcgaagtgacttcctacatcggtggtgaaggtcacaacctgcaggagcactccgtgatcctgatccgtggcggtcgtgttaaagacctcccgggtgttcgttaccacaccgtacgtggtgcgcttgactgctccggcgttaaagaccgtaagcaggctcgttccaagtatggcgtgaagcgtcctaaggcttaaggaggacaatcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaagacaaaggtcctgagacgccaccaccagctccccagctccatcctatct

SEQ ID NO: 2 - DNA, 8293 bp, Artificial Sequence
  Heavy chain knock-in sequence
aattcagacatcatgcagggccagttccctgccctgagctacattagctgggaccagggccagcggttgaggaaccaggcagaggtaaaatggtggtgtgataggaagccaatggcagaggggagggagaagttatgcttatgtcatgctggaatgtaggaaggggaaagagccaggatgtctaggctggagctgatccggctgtctgctctgatggcagcaacaggcctgagcttctctggactcaagaagccagggcaacaaaataaagggggcctagcagagcaaagacactgctagcactgggatcaggaaaacaggacaagactcccgatccaggaggtcatgggagggaaggagaagactacaggggactgtccttgggaaagagtaagggcccactggagggagtgctcaggaagcaagcccattgacaggggagaacaaggctggtggacgtctggatgggcagtaggcagccccaagtcccaggagggagagaagaggcagataggaaaacaggtcaggtctagcagaggcctactgaagtactctcctcaggacagaaccctgaatactggaaaatgcggaactgctgcaggcacaaagaatagctgaggtctaagagtaaaacagactaggggatgagaggaccttaggaagagcctttggctgagcaggaacaagaacaggggaaatcctagggctgacattgccagtggaaacatacaggctggagctctttagtcaggagctccagctgtgatctagacatcaggcaggaagatcaaatctgtcccaacaatacaggggacagaggctcaacctagagtgtgagcatcaggggctgtgcaggagatttcagagctcaggtgcagcagagactagcatggccctggggataaagggaaggatccaagggacaaggggataatcctggggaggtaagggccagcttcgtgacagaaggtggtggtgtccaacttcaagagccctgtgctacaatttaaaaaaaaaaaaaaaggaaagggacttctctgtgtttggcaacacaagtgcgatgcacaggcaggaagatcaaatctgtcccaacaatacaggggacagagggtcaacctacaaaaggaaagaacctggggcagtgtgaagacaacactgtagaagccaaggctgagttcactgagctctcgttagtgagactacacagcaaggaggtggcgggcactgagcagtgaggccccgggaagtgggggtgatggtggtgacggtggtaactgttaagaactgggggaaagaattgtggagaaccaagctaaatagttatgtcaaaccacatgtttaggagcctgggttgacttcatagggagtaggcatggaggctaatctagaggtttgtgtataggcaagaagtgaatcctgacccaagaatagagagtgctaaacggacttagttcaaagacaactgaaaaagacaatgcctgcaaaacaaagctaaggccagagctcttggactatgaagagttcagggaacctaagaacagggaccatctgtgtacaggccaaggccggtagaagcagcctaggaagtgtcaagagccaacgtggctgggtgggcaaagacaggaagggactgttaggctgcagggatgtgccgacttcaatgtgcttcagtattgtccagattgtgtgcagccatatggcccaggtataagaggtttaacagtggaacacagatgcccacatcagacagctggggggcgggggtgaacacagatacccatactggaaagcaggtggggcattttcctaggaacgggactgggctcaatggcctcaggtctcatctggtctggtgatcctgacattgataggcccaaatgttggatatcacctactccatgtagagagtcggggacatgggaagggtgcaaaagagcggccttctagaaggtttggtcctgtcctgtcctgtctgacagtgtaatcacatatactttttcttgtagcctccacacagagcccatccgtcttccccttgacccgctgctgcaaaaacattccctccaatgccacctccgtgactctgggctgcctggccacgggctacttcccggagccggtgatggtgacctgggacacaggctccctcaacgggacaactatgaccttaccagccaccaccctcacgctctctggtcactatgccaccatcagcttgctgaccgtctcgggtgcgtgggccaagcagatgttcacctgccgtgtggcacacactccatcgtccacagactgggtcgacaacaaaaccttcagcggtaagagagggccaagctcagagaccacagttcccaggagtgccaggctgagggctggcagagtgggcaggggttgagggggtgggtgggctcaaacgtgggaacacccagcatgcctggggacccgggccaggacgcgggggcaagaggagggcacacagagctcagagaggccaacaaccctcatgaccaccagctctcccccagtctgctccagggacttcaccccgcccaccgtgaagatcttacagtcgtcctgcgacggcggcgggcacttccccccgaccatccagctcctgtgcctcgtctctgggtacaccccagggactatcaacatcacctggctggaggacgggcaggtcatggacgtggacttgtccaccgcctctaccacgcaggagggtgagctggcctccacacaaagcgagctcaccctcagccagaagcactggctgtcagaccgcacctacacctgccaggtcacctatcaaggtcacacctttgaggacagcaccaagaagtgtgcaggtacgttcccacctgccctggtggccgccacggaggccagagaagaggggcgggtgggcctcacacagccctccggtgtaccacagattccaacccgagaggggtgagcgcctacctaagccggcccagcccgttcgacctgttcatccgcaagtcgcccacgatcacctgtctggtggtggacctggcacccagcaaggggaccgtgaacctgacctggtcccgggccagtgggaagcctgtgaaccactccaccagaaaggaggagaagcagcgcaatggcacgttaaccgtcacgtccaccctgccggtgggcacccgagactggatcgagggggagacctaccagtgcagggtgacccacccccacctgcccagggccctcatgcggtccacgaccaagaccagcggtgagccatgggcaggccggggtcgtgggggaagggagggagcgagtgagcggggcccgggctgaccccacgtctggccacaggcccgcgtgctgccccggaagtctatgcgtttgcgacgccggagtggccggggagccgggacaagcgcaccctcgcctgcctgatccagaacttcatgcctgaggacatctcggtgcagtggctgcacaacgaggtgcagctcccggacgcccggcacagcacgacgcagccccgcaagaccaagggctccggcttcttcgtcttcagccgcctggaggtgaccagggccgaatgggagcagaaagatgagttcatctgccgtgcagtccatgaggcagcaagcccctcacagaccgtccagcgagcggtgtctgtaaatcccggtaaatgacgtactcctgcctccctccctcccagggctccatccagctgtgcagtggggaggactggccagaccttctgtccactgttgcaatgaccccaggaagctacccccaataaactgtgcctgctcagagccccaggtacacccattcttgggagcgggcagggctgtgggcaggtgcatcttggcacagaggaatgggccccccaggaggggcagtgggaggaggtgggcagggctgagtccccccaggagaggcggtgggaggaggtgggcagggctgaggtgccactcatccatctgccttcgtgtcagggttatttgtcaaacagcatatctgcagggactcatcacagctaccccgggccctctctgcccccactctgggtctaccccctccaaggagtccaaagacccaggggaggtcctcagggaaggggcaagggagcccccacagccctctctcttgggggcttggcttctacccccctggacaggagcccctgcacccccaggtatagatgggcacacaggcccctccaggtggaaaaacagccctaagtgaaacccccacacagacacacacgacccgacagccctcgcccaagtctgtgccactggcgttcgcctctctgccctgtcccgccttgccgagtcctggccccagcaccggggccggtggagccgagcccactcacaccccgcagcctccgccaccctgccctgtgggcacaccaggcccaggtcagcagccaggccccctctcctactgccccccaccgccccttggtccatcctgaatcggcccccaggggatcgccagcctcacacacccagtctcgcccactcacgcctcactcaaggcacagctgtgcacacactaggccccatagcaactccacagcaccctgtaccaccaccagggcgccatagacaccccacacgtggtcacacgtggcccacactccgcctctcacgctgcctccagcgaggctactgccaagcccttcctctgagccatacctgggccgctggatcccagagagaaatggagaggccctcacgtggtgtcctccagtccaaccctccctgtcaccctgtcagcagcagcaccccacagccaaacacaggatggatgcgtgggctccatcccccactcacccacaccggaaccccagagcaggctacgtgcccctcacagacctcaaacccacatgtgcatctgacaccccagatccaaacgctccccccggtcatgcacaccaagggcacagcacccaccaaatccacacggaaacacgggcaccgggcaccccatgagcacaaagcccctccatgtctgaagacagtccctgcacaccgtcacagccatacattcagcttcactctcacgtcccagcccacctgcacccagctctgggcctggagcagcagaaagaggtgtgagggcccgaggcgggacctgcacctgctgatgacccgggaccagcaggcagctcacggtgttggggaagggagtggagggcacccagggcaggagccagagggaccaggctggtgggcggggccgggccggggtagggccaggaggcagctctggacacccacaggcctgggctcatagtccacaccaggacagcccctcagagcacccatgcagtgagtcccaggtcttgggagccaggccgcagagctcacgcatccttccgagggccctgagtgaggcggccactgctgtgccgaggggttgggtccttctctggggagggcgtggggtctagagaggcggagtggaggtaaccagaggtcaggagagaagccgtaaggaacagagggaaaatggggccagagtcggggcgcagggacgagaggtcaggagtggtcggcctggctctgggccgttgactgactcgggacctgggtgcccaccctcagggctggctggcggctccgcgcagtcccagagggccccggatagggtgctctgccactccggacagcagcagggactgccgagagcagcaggaggctctgtcccccacccccgctgccactgtggagccgggagggctgactggccaggtcccccagagctggacgtgtgcgtggaggaggccgagggcgaggcgccgtggacgtggaccggcctctgcatcttcgccgcactcttcctgctcagcgtgagctacagcgccgccatcacgctcctcatggtgggcacccacctccaggggcccggccagggcagggggttgggcagagccagcagagcgccctgacccacgccctcccctcaggtgcagcggttcctctcagccacgcggcaggggaggccccagacctccctcgactacaccaacgtcctccagccccacgcctaggccgcgggccacctcctgtaatggcatttcccaggccccgaaggaccctgtccaatatgccaagcagcacaactgagatcacactgtctgctcatctcgctttcctccgaccccgagactcagctactctcaaattttccctctctgaaggaccatgtggacattacattgctccaggccacagccaccaggacctaaaacaccatcacagcagcaccaaagacactggatagacccacaagggcaatagtttcctcaacagtatatccaaactgttgggacaaacgagcaatcactgaagaagtgacaagttcccacaatgtcagtgtccagctgagaagggacaaaaagtggtaccagccctgtccacaccaccttctaattcacaggaatacgtgatagaagaggcaggttgtagatccgaaagatgagacagattttatcaactccagaaagagctgggtccaactgaattattctagcgaccttggcattgtcatgacctgccatgaccttcctccttaacacttcgataaaccctgggatatggaaaatgcctgtgtttctcagggtttgggaaagaaccatccatgttgggattcttgtgtagatcctccttctggtcacagatgcaatacactggattttcaggcaaaggagcaaattcacagacaactctggccctacagccctaagacctagacaccaccatctccttggaattatcaaatttaacacccggcacacaacaaagaaggactgggactttgaggcctttgtgtagccctagagggggcagaggccactgagcagggattgggtgatcacaaggacctcctggagagggacctgaggagcaggttccaattgggccaaagaaagaagaacaacaatagagatgaaggatgctggaaagagccatggtacagcagtcttgtccttcagacatgactcttacagcccaggactcttacagtagctagctggagcagaagtccaagggattaccatgccctagggccacaggctactggagggtggagtgagtctactacacaggtccaatgcctgtttctccattgcttctcagccaatgagaaatcagagtctccacctccaagaaaaaggaaggtggaaatgaaaggtgagcacctgccttcccgtgactggcagaaagatctccacggactcaaggctttgacttcaaacttcccgtagatccctgatgtctttaaggactctgtctgctttgttggttttgtttgtttgttttgtactttgtcaataaaacatttttcaaatattctacaattgctgtgctcttcctatgcaaactggccctggcacttcaaaacatggcacagttaagttgaccagtgggccatgcagagcatactacccctcctggtcctgtgtcctaccagatatcctctaagtgtcccttcactgctgggctccagttctgctgccctgaaccacacaggtcctcagtctgtcctccctatgggcagtttcatcccagccagaagctgccctgtggcccctaggctgcccaggcatggtctcccacaccaaccacacaaactaagaagcctgtcccatacattgacctcaggctcagtgataacatttctatagaaaaaggaaaggaataagaaaaaaaaaactccatattacatgggggctgcctaccttgctgatgcttattgtctcaagcaagtttggagaagttccaaatcaggcactgacagggtgggttctcagccatgctcttcttacctggtaggtgccttctcccccatggcacctcacaggctctccatctgtgtgtgtctgggtcctgatctcttctcataagtacaaagtcaggctggaagaggtacaccctagccctcattataacttaccagtttatgatcctgtctgcaaacatctgaggtccctgtggctcaaatgtcaattagtgagtcttctggggacagaatttagtccacattagctctccctgtggaaagtactgcctacatcctatgcccctccccccatggccacttcccaaggtctcatccatccagcatcagctgtctcaagtctcctccaaagctctgctagttaggtaagggtgagatgtgggtgtaactcattcaggggcaagctcacattgtataggccatggctgtcaggaatgtcattaaggctgtcagcacagagaagacacttattccaggttgatctgagcaggtgctgagc

SEQ ID NO: 3 - DNA, 1613 bp, Artificial Sequence
  Heavy chain selection marker
tatctcagagttcaagtgtacagtccccgaagcaagagtcttgccttcttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttccactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcagcacgtgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacgatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgacgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgcccccgattcgcagcgcatcgccttctatcgccttcttgacgagttcttctgagcgggactctggggttcgaataaagaccgaccaagcgacgtctgagagctccctggcgaattcggtaccaataaaagagctttattttcatgatctgtgtgttggtttttgtgtgcggcgcgttctgttgcatcctgacaatagcagttttcctcaatggcagtttaagcct

SEQ ID NO: 4 - DNA, 1419 bp, Artificial Sequence; identical to SEQ ID NO: 1
  Light chain counter selection marker

SEQ ID NO: 5 - DNA, 423 bp, Artificial Sequence
  Light chain knock-in sequence
tggagcccttccttgttacttcataccatcctctgtgcttccttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagacaaaggtcctgagacgccaccaccagctccccagctccatcctatct

SEQ ID NO: 6 - DNA, 1716 bp, Artificial Sequence
  Light chain selection marker
ttcttcccctaagtcgtcaaagtcctgaagggggacagtctttacaagcagggcgaattgggccgcataacttcgtatagcatacattatacgaagttatattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttccactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcagcacgtgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacgatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgacgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgcccccgattcgcagcgcatcgccttctatcgccttcttgacgagttcttctgagcgggactctggggttcgaataaagaccgaccaagcgacgtctgagagctccctggcgaattcggtaccaataaaagagctttattttcatgatctgtgtgttggtttttgtgtgcggcgcgataacttcgtatagcatacattatacgaagttatctcgatagcttgagtacatgttctgtaatctgattcaacctacccagtaaacttggcgaagcaaag

SEQ ID NO: 7 - DNA, 937 bp, Artificial Sequence
  Dig-labelled probe
gggataatcctggggaggtaagggccagcttcgtgacagaaggtggtggtgtccaacttcaagagccctgtgctacaatttaaaaaaaaaaaaaaaggaaagggacttctctgtgtttggcaacacaagtgcgatgcacaggcaggaagatcaaatctgtcccaacaatacaggggacagagggtcaacctacaaaaggaaagaacctggggcagtgtgaagacaacactgtagaagccaaggctgagttcactgagctctcgttagtgagactacacagcaaggaggtggcgggcactgagcagtgaggccccgggaagtgggggtgatggtggtgacggtggtaactgttaagaactgggggaaagaattgtggagaaccaagctaaatagttatgtcaaaccacatgtttaggagcctgggttgacttcatagggagtaggcatggaggctaatctagaggtttgtgtataggcaagaagtgaatcctgacccaagaatagagagtgctaaacggacttagttcaaagacaactgaaaaagacaatgcctgcaaaacaaagctaaggccagagctcttggactatgaagagttcagggaacctaagaacagggaccatctgtgtacaggccaaggccggtagaagcagcctaggaagtgtcaagagccaacgtggctgggtgggcaaagacaggaagggactgttaggctgcagggatgtgccgacttcaatgtgcttcagtattgtccagattgtgtgcagccatatggcccaggtataagaggtttaacagtggaacacagatgcccacatcagacagctggggggcgggggtgaacacagatacccatactggaaagcaggtggggcattttcctaggaacgggactgggctcaatggcctcaggtctcatctggtctggtgatcctgaca

SEQ ID NO: 8 - DNA, 74 bp, Artificial Sequence
  Heavy chain rpsl-neo cloning forward primer
gtcctgtcctgtcctgtctgacagtgtaatcacatatactttttcttgtaggcctggtgatgatggcgggatcg

SEQ ID NO: 9 - DNA, 74 bp, Artificial Sequence
  Heavy chain rpsl-neo cloning reverse primer
tattggacagggtccttcggggcctgggaaatgccattacaggaggtggctcagaagaactcgtcaagaaggcg

SEQ ID NO: 10 - DNA, 26 bp, Artificial Sequence
  Mouse Cgamma1 cloning forward primer
gaattcagacatcatgcagggccagt

SEQ ID NO: 11 - DNA, 26 bp, Artificial Sequence
  Mouse Cgamma1 cloning reverse primer
atcgattacaagaaaaagtatatgtg

SEQ ID NO: 12 - DNA, 26 bp, Artificial Sequence
  Human Cepsilon cloning forward primer
atcgatgcctccacacagagcccatc

SEQ ID NO: 13 - DNA, 18 bp, Artificial Sequence
  Human Cepsilon cloning reverse primer
aaagctgggcctggtgga

SEQ ID NO: 14 - DNA, 20 bp, Artificial Sequence
  Mutagenesis forward primer
gcctccacacagagcccatc

SEQ ID NO: 15 - DNA, 20 bp, Artificial Sequence
  Mutagenesis reverse primer
tacaagaaaaagtatatgtg

SEQ ID NO: 16 - DNA, 24 bp, Artificial Sequence
  Mouse Cgamma1 cloning forward primer
ccgcgggccacctcctgtaatggc

SEQ ID NO: 17 - DNA, 24 bp, Artificial Sequence
  Mouse Cgamma1 cloning reverse primer
ctcgagctcagcacctgctcagat

SEQ ID NO: 18 - DNA, 70 bp, Artificial Sequence
  Heavy chain neo cloning forward primer
tgggtccaactgaattattctagcgaccttggcattgtcatgacctgccagggcgaattgggccgcataa

SEQ ID NO: 19 - DNA, 70 bp, Artificial Sequence
  Heavy chain neo cloning reverse primer
aggcattttccatatcccagggtttatcgaagtgttaaggaggaaggtcatactcaagctatcgagataa

SEQ ID NO: 20 - DNA, 74 bp, Artificial Sequence
  Light chain rpsl-neo cloning forward primer
atggagcccttccttgttacttcataccatcctctgtgcttccttcctcaggcctggtgatgatggcgggatcg

SEQ ID NO: 21 - DNA, 74 bp, Artificial Sequence
  Light chain rpsl-neo cloning reverse primer
agataggatggagctggggagctggtggtggcgtctcaggacctttgtcttcagaagaactcgtcaagaaggcg

SEQ ID NO: 22 - DNA, 69 bp, Artificial Sequence
  Mouse Ckappa cloning forward primer
atggagcccttccttgttacttcataccatcctctgtgcttccttcctcaggaactgtggctgcaccat

SEQ ID NO: 23 - DNA, 69 bp, Artificial Sequence
  Mouse Ckappa cloning reverse primer
agataggatggagctggggagctggtggtggcgtctcaggacctttgtctctaacactctcccctgttg

SEQ ID NO: 24 - DNA, 70 bp, Artificial Sequence
  Mouse neo cloning forward primer
ttcttcccctaagtcgtcaaagtcctgaagggggacagtctttacaagcagggcgaattgggccgcataa

SEQ ID NO: 25 - DNA, 70 bp, Artificial Sequence
  Mouse neo cloning reverse primer
ctttgcttcgccaagtttactgggtaggttgaatcagattacagaacatgtactcaagctatcgagataa

SEQ ID NO: 26 - DNA, 20 bp, Artificial Sequence
  Heavy chain rpsl-neo screening forward primer
gcctcaggtctcatctggtc

SEQ ID NO: 27 - DNA, 20 bp, Artificial Sequence
  Heavy chain rpsl-neo screening reverse primer
cttggaacgagcctgcttac

SEQ ID NO: 28 - DNA, 20 bp, Artificial Sequence
  Heavy chain rpsl-neo screening forward primer
gcctcaggtctcatctggtc

SEQ ID NO: 29 - DNA, 20 bp, Artificial Sequence
  Heavy chain rpsl-neo screening reverse primer
ggttttgttgtcgacccagt

SEQ ID NO: 30 - DNA, 20 bp, Artificial Sequence
  Human Cepsilon screening forward primer
cactgtgcaaccaaacatcc

SEQ ID NO: 31 - DNA, 20 bp, Artificial Sequence
  Heavy chain neo screening reverse primer
tgtccatctgcacgagacta

SEQ ID NO: 32 - DNA, 18 bp, Artificial Sequence
  Light chain neo screening forward primer
ccctccaatcgggtaact

SEQ ID NO: 33 - DNA, 18 bp, Artificial Sequence
  Light chain neo screening reverse primer
gtttggagcaccgcaaca

SEQ ID NO: 34 - DNA, 20 bp, Artificial Sequence
  Mouse Cgamma1 screening forward primer
cctggccccattgttcctta

SEQ ID NO: 35 - DNA, 20 bp, Artificial Sequence
  Light chain neo screening forward primer
tccactacatggcagtcctt

SEQ ID NO: 36 - DNA, 20 bp, Artificial Sequence
  Heavy chain genotyping forward primer
gaaggtttggtcctgtcctg

SEQ ID NO: 37 - DNA, 22 bp, Artificial Sequence
  Heavy chain genotyping reverse primer
gctgctcagagtgtagaggtca

SEQ ID NO: 38 - DNA, 20 bp, Artificial Sequence
  Heavy chain genotyping reverse primer
ggttttgttgtcgacccagt

SEQ ID NO: 39 - DNA, 20 bp, Artificial Sequence
  Light chain genotyping forward primer
ggcagtgaacgacaaaatgg

SEQ ID NO: 40 - DNA, 20 bp, Artificial Sequence
  Light chain genotyping reverse primer
cctggccccattgttcctta

SEQ ID NO: 41 - DNA, 18 bp, Artificial Sequence
  Light chain genotyping reverse primer
gtttggagcaccgcaaca

SEQ ID NO: 42 - DNA, 20 bp, Artificial Sequence
  Dig-labelled probe forward primer
gggataatcctggggaggta

SEQ ID NO: 43 - DNA, 20 bp, Artificial Sequence
  Dig-labelled probe reverse primer
tgtcaggatcaccagaccag

SEQ ID NO: 44 - DNA, 20 bp, Artificial Sequence
  Mouse Cgamma1 forward primer
atggcaaggagttcaaatgc

SEQ ID NO: 45 - DNA, 20 bp, Artificial Sequence
  Mouse Cgamma1 reverse primer
tttatccttggccatctgct

SEQ ID NO: 46 - DNA, 18 bp, Artificial Sequence
  Human Cepsilon forward primer
tctaccacgcaggagggt

SEQ ID NO: 47 - DNA, 18 bp, Artificial Sequence
  Human Cepsilon reverse primer
ctgtcctcaaaggtgtga

SEQ ID NO: 48 - DNA, 19 bp, Artificial Sequence
  Mouse beta-actin forward primer
gactacctatgaagatcct

SEQ ID NO: 49 - DNA, 20 bp, Artificial Sequence
  Mouse beta-actin reverse primer
ccacatctgctggaaggtgg